(19) United States
(12) Patent Application Publication
(10) Pub. No.: US 2022/0218212 A1
(43) Pub. Date: Jul. 14, 2022

(54) CARDIAC OUTPUT MEASUREMENT DEVICE AND CARDIAC OUTPUT MEASUREMENT METHOD

(71) Applicant: TERUMO KABUSHIKI KAISHA, Tokyo (JP)

(72) Inventors: SHINICHIRO SUDA, Kanagawa (JP); XIAOWEI LU, Kanagawa (JP); KEI HONDA, Kanagawa (JP)

(21) Appl. No.: 17/707,212

(22) Filed: Mar. 29, 2022

Related U.S. Application Data
(63) Continuation of application No. PCT/JP2020/037130, filed on Sep. 30, 2020.

(30) Foreign Application Priority Data
Sep. 30, 2019 (JP) 2019-178944

(51) Int. Cl. A61B 5/029; A61B 5/0507; A61B 5/08; A61B 5/113; A61B 5/00

(52) U.S. Cl. CPC A61B 5/029; A61B 5/0507; A61B 2562/0219; A61B 5/113; A61B 5/7225; A61B 5/0826

(57) ABSTRACT

A cardiac output measurement device according to at least one embodiment of the present disclosure includes a cardiac waveform measurement unit that measures a waveform of microwaves transmitted through a living body; a respiratory waveform measurement unit that measures a waveform during breathing or a waveform during apnea of the living body; and a cardiac output calculation unit that calculates a waveform for obtaining a cardiac output of the living body from the waveform of the microwaves by using the waveform during breathing or the waveform during apnea.

### CROSS REFERENCE TO RELATED APPLICATIONS

The present disclosure is a continuation of and claims benefit to PCT Application No. PCT/JP2020/037130 filed on Sep. 30, 2020, entitled “CARDIAC OUTPUT MEASUREMENT DEVICE AND CARDIAC OUTPUT MEASUREMENT METHOD” which claims priority to Japanese Patent Application No. 2019-178944 filed on Sep. 30, 2019. The entire disclosure of the applications listed above are hereby incorporated herein by reference, in their entirety, for all that they teach and for all purposes.

### BACKGROUND

The present disclosure relates to a cardiac output measurement device and a cardiac output measurement method which are capable of measuring a cardiac output in a state in which a subject is breathing.

In order to know whether or not a heart of a subject (e.g., a patient) is functioning normally, it is important to measure a cardiac output indicating how much blood is being pumped from the heart.

Examination of heart failure, follow-up observation after heart surgery, verification of an effect of medication for a heart disease, and the like can be performed by measuring the cardiac output. Examples of a device that measures the cardiac output include various devices as disclosed in Japanese Patent No. JP2016-202516A and International Publication No. WO2018/194093A1.

However, typically, measurement of the cardiac output is performed in a state in which a subject is breathing, and thus an influence of breathing cannot be ignored for accurate measurement of the cardiac output. The devices disclosed in JP2016-202516A and International Publication No. WO2018/194093A1 do not take into account the influence of breathing, and thus it is difficult to easily perform high-accuracy measurement of the cardiac output in a hospital, a care facility, and the like.

### SUMMARY

The present disclosure provides a cardiac output measurement device and a cardiac output measurement method which are capable of measuring a cardiac output in a state in which a subject is breathing.

In accordance with at least one embodiment of the present disclosure, there is provided a cardiac output measurement device including: a first measurement unit; a second measurement unit; and a calculation unit.

The first measurement unit measures a waveform of microwaves transmitted through a living body. The second measurement unit measures a waveform during breathing or a waveform during apnea of the living body. The calculation unit calculates a waveform for obtaining a cardiac output of the living body from the waveform of the microwaves by using the waveform during breathing or the waveform during apnea.

In accordance with at least one embodiment of the present disclosure, there is provided a cardiac output measurement method including: measuring a cardiac waveform composed of an apnea component waveform from microwaves transmitted through a living body; calculating a frequency of the apnea component waveform; measuring a waveform during breathing or a waveform during apnea from a displacement of a body surface of the living body; calculating a frequency of the waveform during breathing; measuring a cardiac waveform including both a respiratory component waveform and the apnea component waveform from the microwaves transmitted through the living body; shaping the cardiac waveform by using frequencies of the apnea component waveform and the waveform during breathing; and calculating a cardiac output from the shaped cardiac waveform.

According to embodiments of the present disclosure, an influence of breathing of a subject is removed, and thus accurate measurement of a cardiac output can be performed even in a state in which the subject is breathing.

### BRIEF DESCRIPTION OF THE DRAWINGS

FIG. 1 is a block diagram of a cardiac output measurement device according to at least one embodiment of the present disclosure;

FIG. 2A is a view illustrating a waveform of microwaves measured by a cardiac waveform measurement unit according to at least one embodiment of the present disclosure;

FIG. 2B is a view illustrating a respiratory waveform measured by a respiratory waveform measurement unit according to at least one embodiment of the present disclosure;

FIG. 2C is a view illustrating a cardiac waveform after application with a filter suitable for a frequency of a respiratory waveform according to at least one embodiment of the present disclosure;

FIG. 2D is a view illustrating a cardiac waveform after application with a filter suitable for a heart rate according to at least one embodiment of the present disclosure;

FIG. 3 is an operation flowchart for calculating the heart rate by a cardiac output measurement device according to at least one embodiment of the present disclosure;

FIG. 4 is an operation flowchart for calculating a frequency of the respiratory waveform by a cardiac output measurement device according to at least one embodiment of the present disclosure;

FIG. 5A is a view illustrating an apnea component waveform measured by the cardiac waveform measurement unit according to at least one embodiment of the present disclosure;

FIG. 5B is a view illustrating a respiratory waveform measured by the respiratory waveform measurement unit according to at least one embodiment of the present disclosure;

FIG. 6 is an operation flowchart for calculating a cardiac output by the cardiac output measurement device of this embodiment according to at least one embodiment of the present disclosure;

FIG. 7A is a view illustrating a cardiac waveform including a respiratory component waveform and an apnea component waveform measured by the cardiac waveform measurement unit according to at least one embodiment of the present disclosure; and

FIG. 7B is a view illustrating an example of a shaped cardiac waveform, which is a waveform for obtaining a cardiac output, that is used to calculate a cardiac output by a cardiac output calculation unit according to at least one embodiment of the present disclosure.

### DETAILED DESCRIPTION

Hereinafter, a cardiac output measurement device and a cardiac output measurement method will be described in accordance with embodiments of the present disclosure.

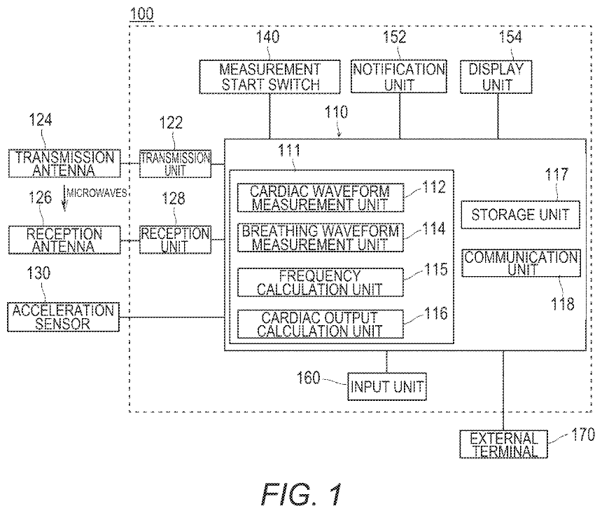

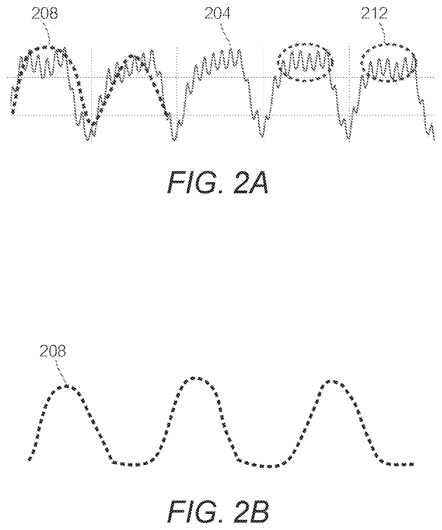

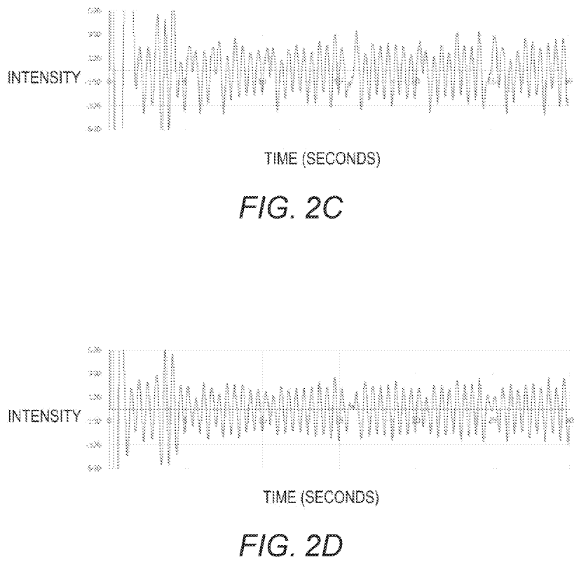

FIG. 1 is a block diagram of the cardiac output measurement device according to at least one embodiment of the present disclosure. FIG. 2A is a view illustrating a reception waveform of microwaves 204 received by a reception unit. A cardiac output measurement device 100 includes a control unit 110, a transmission unit 122, a reception unit 128, a measurement start switch 140, a notification unit 152, a display unit 154, and an input unit 160.

The control unit 110 calculates a cardiac output of a subject (e.g., a patient) or, in order words, the amount of blood (in, for example, liters/minute) pumped from a left ventricle of a heart of the subject per unit time by using a waveform of microwaves which are transmitted through a chest of the subject or patient (e.g., a living body) and are received by the reception unit 128.

In a case where the microwaves are transmitted through the heart of the subject, since the microwaves are absorbed by blood, the waveform of the microwaves is further attenuated in a diastolic phase in which blood flows into the heart in comparison to a systolic phase in which blood flows out from the heart. The cardiac output can be calculated from an attenuation amount (e.g., an amplitude) of the waveform of the microwaves. In measurement of the cardiac output by using the microwaves, there are an advantage that measurement of the cardiac output can be performed in a non-invasive manner, and an advantage that a size of a device used to measure the cardiac output can be reduced. To carry out medical treatment of heart failure, follow-up observation after heart surgery, verification of an effect of medication for a heart disease, and the like, it is important that a measurement device is a non-invasive type and is small in size, and the cardiac output can be measured anytime, anywhere, and any number of times using embodiments discussed herein. Accordingly, it is very important to accurately calculate the attenuation amount of the waveform of the microwaves for accurate calculation of the cardiac output.

When measuring the cardiac output of the subject, since microwaves are emitted from the heart of the subject, an influence of breathing of the subject cannot be ignored. The subject may breathe at different rates and with different intensities, such as shallow breathing, deep breathing, slow breathing, fast breathing, regular breathing, and irregular breathing, depending on a measurement environment and a condition. A relative position between a transmission antenna and a reception antenna disposed on a body surface of a chest may vary based on the different types of patient breathing, and such breathing hinders accurate calculation of the attenuation amount of the waveform of the microwaves. In addition, since the microwaves are also absorbed by the lungs, a variation in the capacity of the lungs due to breathing also hinders accurate calculation of the attenuation amount of the waveform of the microwaves. The control unit 110 accurately calculates the attenuation amount of the waveform of the microwaves by removing the influence due to breathing of the subject. The control unit 110 is provided with various constituent elements for accurately calculating the attenuation amount of the waveform of the microwaves, as described in further detail below.

In the heart failure, signs or symptoms are likely to appear in a breathing state, and there are characteristics in which breathing becomes difficult when the heart failure gets worse, and breathing can be performed normally when the patient improves or recovers. Since the breathing rate and the depth and pattern of breathing vary from moment to moment in accordance with a variation of signs or symptoms, cardiac output measurements in which measurement accuracy does not depend on the breathing state is very important in medical treatment of the heart failure.

During hospitalization, many patients who exit, or who have gone through, cardiac surgery and many patients with heart failure are managed in a centralized management area with abundant monitoring devices such as an Intensive Care Unit (ICU) and a Cardiac Care Unit (CCU), where the patients can be monitored by medical staff. In contrast, some patients are managed in a general ward area, which may not have the proper monitoring devices or medical staff as the patients recover, and are eventually discharged from a hospital. A device capable of evaluating a breathing state may exist in the centralized management area, but typically, the device capable of evaluating the breathing state may not exist in the general ward area. It is important to continuously manage the cardiac output for patients after cardiac surgery or patients with heart failure from hospitalization to discharge from a hospital. Accordingly, realizing cardiac output measurement in which measurement accuracy does not depend on a breathing state is very important to enable a measurement of the cardiac output regardless of a location such as the ICU, the CCU, and the general ward, and to realize cardiac output management in the entirety of the patient flow during hospitalization.

Heart failure is a disease characterized by declining condition and repeated re-hospitalizations, and thus it is necessary to determine the cardiac output not only in a hospital environment but also in other environments, such as at home, in a nursing facility, and in a family clinic. Accordingly, it is important to easily perform high-accuracy measurement of the cardiac output anywhere regardless of breathing.

The transmission unit 122 receives an instruction from the control unit 110, and transmits a signal for emitting microwaves having a predetermined frequency from a transmission antenna 124. The microwave frequency may be adjusted to ensure the clearest possible waveforms are obtained. In some embodiments, microwaves having a frequency of 0.4 to 1.00 Gigahertz (GHz) are used.

The reception unit 128 amplifies a signal of the microwaves received by a reception antenna 126. The transmission antenna 124 and the reception antenna 126 may be positioned on either side of the chest of the subject. In some embodiments, the transmission antenna 124 is disposed on the back side of the subject, and the reception antenna 126 is disposed on the chest side of the subject. In other embodiments, the transmission antenna 124 is disposed on the chest side of the subject, and the reception antenna 126 is disposed on the back side of the subject. In addition, the transmission antenna 124 and the reception antenna 126 may be disposed to come into close contact with a body surface of the subject, or may be disposed to be spaced apart from the body surface of the subject by a constant distance. It is preferable that the transmission antenna 124 and the reception antenna 126 are disposed at the periphery of the heart of the subject, particularly, with the left ventricle interposed therebetween. Accordingly, the reception antenna 126 receives a waveform of microwaves which are emitted from the transmission antenna 124 and are transmitted through the chest of the subject, for example, as illustrated in FIG. 2A. The subject is irradiated with the microwaves during breathing. Accordingly, the waveform of the microwaves illustrated in FIG. 2A includes both a waveform during breathing 208 which is obtained when the subject is breathing (when the chest vertically moves), and a waveform during apnea 212 which is obtained when breathing is stopped for a moment (in other words, when only a heartbeat is measured).

The measurement start switch 140 is configured to give an instruction for start of measurement for the cardiac output by a user such as medical workers including a doctor and a nurse. A specific aspect of the measurement start switch 140 is not particularly limited as long as the measurement start switch 140 is a switch capable of switching on and off. For example, a mechanical switch such as a toggle type or a button type, or an electronic switch displayed on a display screen.

The notification unit 152 gives a notification of a message promoting stoppage of breathing of the subject. In other words, the notification unit 152 may instruct, through a message or notification appearing on a display, the patient or other subject to stop breathing. In a state in which the subject is breathing, as illustrated in FIG. 2A, a reception waveform of the microwaves 204 received by the reception unit 128 becomes a reception waveform of microwaves in which a waveform during breathing 208 and a waveform during apnea 212 are mixed. Therefore, when desiring to measure a reception waveform of microwaves only during apnea of the subject, the notification unit 152 is caused to notify the subject of a message that promotes stoppage of breathing. The notification unit 152 may give a notification of the message promoting stoppage of breathing with sound or light, or by displaying characters on a screen.

The display unit 154 displays various waveforms calculated by the control unit 110, and a calculated cardiac output. The display unit 154 is a display using liquid crystal or organic electroluminescent (EL) display.

An input unit 160 is configured to allow users such as medical workers to input information of the subject (e.g., a sex, an age, a name, a weight, a height, or the like of the subject) and to input measurement contents to the control unit 110. The input unit 160 can be or comprise any pointing device such as a press button, a keyboard, and a mouse or the entirety thereof, or a partial combination thereof. In some embodiments, the input unit 160 is provided in the cardiac output measurement device 100, but may be externally attached to the cardiac output measurement device 100.

An external terminal 170 is configured to communicate with the cardiac output measurement device 100 through a communication unit 118. The external terminal 170 may be or comprise a known tablet, a known personal computer, or the like.

The control unit 110 includes a cardiac waveform measurement unit 112, a respiratory waveform measurement unit 114, a frequency calculation unit 115, a cardiac output calculation unit 116, a storage unit 117, and the communication unit 118. The cardiac waveform measurement unit 112, the respiratory waveform measurement unit 114, the frequency calculation unit 115, and the cardiac output calculation unit 116 are disposed in a processor 111.

In some embodiments, the processor 111 may correspond to one or more computer processing devices. For example, the processor 111 and/or one or more components thereof (e.g., the cardiac waveform measurement unit 112, the respiratory waveform measurement unit 114, the frequency calculation unit 115, the cardiac output calculation unit 116, etc.) may be provided as silicon, an Application-Specific Integrated Circuit (“ASIC”), as a Field Programmable Gate Array (“FPGA”), any other type of Integrated Circuit (“IC”) chip, a collection of IC chips, and/or the like. In some embodiments, the processor 111 and/or one or more components thereof may be provided as a Central Processing Unit (“CPU”), a microprocessor, or a plurality of microprocessors that are configured to execute the instructions sets. In some embodiments, the components of the processor 111 (e.g., the cardiac waveform measurement unit 112, the respiratory waveform measurement unit 114, the frequency calculation unit 115, the cardiac output calculation unit 116, etc.) may be embodied as a virtual processor(s) executing on one or more physical processors. The execution of a virtual processor may be distributed over a number of physical processors or one physical processor may execute one or more virtual processors. Virtual processors are presented to a process as a physical processor for the execution of the process while the specific underlying physical processor(s) may be dynamically allocated before or during the execution of the virtual processor wherein the instruction stack and pointer, register contents, and/or other values maintained by the virtual processor for the execution of the process are transferred to another physical processor(s). As a benefit, the physical processors may be added, removed, or reallocated without affecting the virtual processors execution of the processes. Additionally or alternatively, the physical processor(s) may execute a virtual processor to provide an alternative instruction sets as compared to the instruction set of the virtual processor (e.g., an “emulator”). As a benefit, a process compiled to run a processor having a first instruction set (e.g., Virtual Address Extension (“VAX”)) may be executed by a processor executing a second instruction set (e.g., Intel® 9xx chipset code) by executing a virtual processor having the first instruction set (e.g., VAX emulator).

FIG. 2A is a view illustrating a waveform of microwaves which is measured by the cardiac waveform measurement unit 112. The cardiac waveform measurement unit 112 functions as a first measurement unit that measures a waveform of microwaves transmitted through the subject.

The cardiac waveform measurement unit 112 measures a waveform of microwaves comprising a composite waveform of a respiratory component waveform 208 and an apnea component waveform 212 as illustrated in FIG. 2A from the microwaves transmitted through the subject. In the example illustrated in FIG. 2A, a frequency of the respiratory component waveform 208 is lower than a frequency of the apnea component waveform 212, and an entire shape of the composite waveform is obtained by the respiratory component waveform, and the apnea component waveform 212 is shown as a fine unevenness. In the example illustrated in FIG. 2A, variations in the waveform of the microwaves in the apnea component waveform 212 may be due to inflow and outflow of blood to and from the heart. The cardiac waveform measurement unit 112 measures a reception waveform of microwaves amplified by the reception unit 128 as illustrated in FIG. 2A. The waveform of the microwaves includes a respiratory component waveform 208 while the subject is breathing and an apnea component waveform 212 while the subject is not breathing. Additionally, the apnea component waveform 212 is included in the respiratory component waveform.

FIG. 2B is a view illustrating a respiratory waveform measured by the respiratory waveform measurement unit 114. The respiratory waveform measurement unit 114 functions as a second measurement unit that measures a waveform during breathing of the subject or a waveform during apnea (vertical movement of the chest).

The respiratory waveform measurement unit 114 measures the waveform during breathing or the waveform during apnea from a displacement of a body surface of the chest of the subject. An acceleration sensor 130 mounted on the body surface of the subject is connected to the respiratory waveform measurement unit 114. The acceleration sensor 130 is mounted on the chest of the subject, and detects a vertical movement of the chest of the subject while the subject is breathing as a positional displacement. In FIG. 2B, a situation in which the waveform is rising represents that the subject is inhaling, a situation in which the waveform is falling represents that the subject is exhaling, and the vicinity of the top and the vicinity of the bottom of the waveform represent that the subject is holding his breath. A waveform while the subject is inhaling and a waveform while the subject is exhaling represent a waveform during breathing, and a waveform while the subject is holding his breath represents a waveform during apnea. Note that, in some embodiments, the acceleration sensor 130 may detect the vertical displacement of the chest of the subject. However, for example, a sensor that detects a positional displacement from a pressure such as a pressure sensor, or a distance measurement sensor such as a laser sensor that detects a positional displacement from a distance may be used as long as the positional displacement can be detected. In addition, in a case of using the acceleration sensor, a situation in which a waveform is falling may represent that the subject is inhaling and a situation in which a waveform is rising may represent that the subject is exhaling.

FIG. 2C is a view illustrating a cardiac waveform after application with a filter suitable for a frequency of a waveform during breathing. FIG. 2D is a view illustrating a cardiac waveform after application with a filter suitable for a frequency of an apnea component waveform 212. The frequency calculation unit 115 and the cardiac output calculation unit 116 function as a calculation unit that calculates a waveform for obtaining a cardiac output of a heart of the subject from a waveform of microwaves which is measured by the cardiac waveform measurement unit 112 by using a waveform during breathing or a waveform during apnea which is measured by the respiratory waveform measurement unit 114.

The frequency calculation unit 115 calculates a frequency of an apnea component waveform 212 in a reception waveform of microwaves 204 which is measured by the cardiac waveform measurement unit 112 as illustrated in FIG. 2A, and a frequency of a waveform during breathing which is measured by the respiratory waveform measurement unit 114 as illustrated in FIG. 2B. The calculation of the frequency of the apnea component waveform 212 in the reception waveform of the microwaves by the frequency calculation unit 115, that is, a heart rate due to a waveform variation of the microwaves due to flowing-in and flowing-out of blood to and from the heart, and the frequency of the waveform during breathing, that is, a breathing frequency is performed by using a known method that is typically used, for example, a method in which a frequency of a waveform is calculated by the number of times of crossing of a constant threshold value by a voltage per unit time, or the like.

The heart rate is frequently equal to the heart rate.

As a method of measuring the frequency of the apnea component waveform 212 in the reception waveform of the microwaves to obtain the frequency of the waveform component caused by heartbeat, a method of emitting the microwaves in a state in which breathing of the subject is stopped to obtain the reception waveform of the microwaves can be exemplified. A waveform obtained by stopping breathing of the subject during emission of the microwaves becomes a waveform composed of the apnea component waveform 212 in FIG. 2A. A heart rate can be obtained by calculating the frequency of the waveform. In addition, even in a case where the microwaves are emitted in a state in which the subject is breathing, or a case where the subject who is instructed to stop breathing suddenly breathes and thus a reception waveform in which the breathing component 208 and the apnea component 212 are mixed is obtained as illustrated in FIG. 2A, the heart rate can be obtained by selecting a waveform to be calculated on the basis of time information indicating when the subject breathed, or the heart rate can also be obtained by performing calculation only for a waveform in which an amplitude intensity of a reception waveform is equal to or less than a constant value.

The cardiac output calculation unit 116 shapes the cardiac waveform corresponding to a heartbeat as illustrated in FIG. 2C or FIG. 2D by using the frequencies of the apnea component waveform 212 and the waveform during breathing in the cardiac waveform which are calculated by the frequency calculation unit 115, and sets the cardiac waveform as illustrated in FIG. 2C or FIG. 2D as a waveform for obtaining a cardiac output and calculates the cardiac output. Note that, calculation of the cardiac output is performed by using a typical known method. When shaping the cardiac waveform, the cardiac output calculation unit 116 generates a filter suitable for a frequency of the waveform during breathing as illustrated in FIG. 2B. A specific method of generating the filter will be described later. When the cardiac waveform is shaped by applying the filter that is suitable for the frequency during breathing and is generated by the cardiac output calculation unit 116 to the reception waveform of the microwaves as illustrated in FIG. 2A, a cardiac waveform from which the respiratory waveform component is removed to a certain extent as illustrated in FIG. 2C is obtained. In addition, when shaping the cardiac waveform, the cardiac output calculation unit 116 generates a filter suitable for a frequency of a waveform component caused by a heartbeat. A specific method of generating the filter will be described later. When further applying the filter that is generated by the cardiac output calculation unit 116 and is suitable for the frequency of the waveform component caused by the heartbeat to the reception waveform of the microwaves as illustrated in FIG. 2A or the cardiac waveform illustrated in FIG. 2C, a waveform for obtaining a cardiac output as illustrated in FIG. 2D, specifically, a waveform of which an amplitude is accurately reproduced for obtaining the cardiac output is obtained. The cardiac output calculation unit 116 calculates the amount of blood that is pumped per unit time by the heart of the subject, that is, the cardiac output from the waveform for obtaining the cardiac output illustrated in FIG. 2D. The cardiac output can also be calculated from an amplitude variation in any of FIG. 2C and FIG. 2D, but when using the cardiac waveform after application with both the filter suitable for the breathing frequency and the filter suitable for the heart rate as illustrated in FIG. 2D, the cardiac output can be more accurately calculated. Note that, in some embodiments, the filter suitable for the heart rate is applied after applying the filter suitable for the breathing frequency, but the filter suitable for the breathing frequency may be applied after applying the filter suitable for the heart rate.

The storage unit 117 stores a filter coefficient calculation formula and the filters for generating the filter suitable for the frequency of the waveform during breathing, and the filter suitable for the frequency of the apnea component waveform 212 included in the reception waveform of the microwaves. The cardiac output calculation unit 116 generates the filter suitable to filter out the frequency of the waveform during breathing as illustrated in FIG. 2B, and the filter suitable to filter out the frequency of the apnea component waveform 212 included in the reception waveform of the microwaves as illustrated in FIG. 2A from filter coefficient calculation formulae (or formulas) and frequencies stored in the storage unit 117. Examples of the filters include digital filters such as a low-pass filter and a bandpass filter.

Accordingly, the cardiac output calculation unit 116 generates the filter suitable to filter out the breathing frequency and the filter suitable to filter out the heart rate from the filter coefficient calculation formulae stored in the storage unit 117 by using the breathing frequency and the heart rate which are calculated by the frequency calculation unit 115.

The reason why the suitable filters are generated by using the frequency of the heartbeat waveform, and the frequency of the respiratory waveform is as follows. The subject may breath at different rates and/or with different intensities, such as shallow breathing, deep breathing, slow breathing, fast breathing, regular breathing, and irregular breathing, depending on a measurement environment and a patient condition (e.g., illness). The heartbeat or a heart rate of the subject may be different depending on the condition. In a waveform shaping method that is generally used, it is assumed that a waveform that is a waveform shaping target is not affected by an environment or the like and is approximately the same waveform. However, breathing of the subject and/or the heart rate can vary depending on the environment, the condition, or the like. As described above, in order to accurately obtain the cardiac output from a waveform of which a frequency may greatly vary, it is necessary to generate each suitable filter by using a frequency of a waveform to filter or remove inconsistencies in breathing from the cardiac waveform.

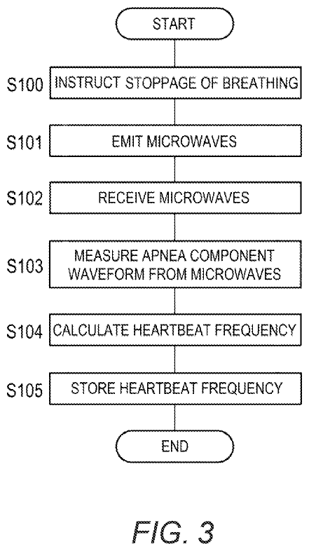

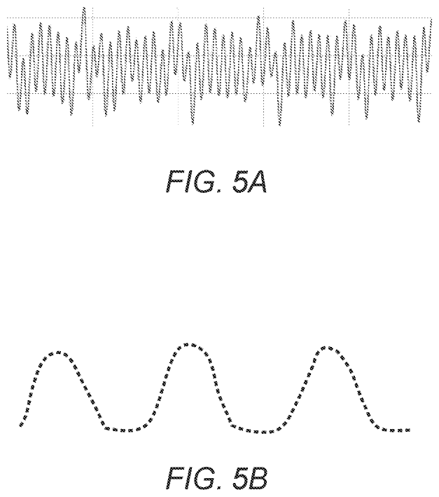

Turning next to the operation of the cardiac output measurement device 100, FIG. 3 is an operation flowchart for calculating the heart rate from the apnea component waveform 212 by the cardiac output measurement device 100 of this embodiment. FIG. 5A is a view illustrating a heartbeat component waveform measured by the cardiac waveform measurement unit 112. In the operation flowchart, microwaves are emitted in a state in which breathing of a subject is stopped, a respiratory component waveform in a reception waveform of microwaves in FIG. 2A is set not to be measured, and a heart rate is calculated from the reception waveform of the microwaves which includes the apnea component waveform 212.

First, the cardiac output measurement device 100 instructs the patient to “stop breathing” (S100). The instruction is performed by giving a notification of a message for promoting stoppage of breathing of a living body by the notification unit 152. Notification of the message for promoting stoppage of breathing may be given with sound or light, or may be given by displaying characters on a screen.

Next, the control unit 110 instructs the transmission unit 122 to output microwaves, and the transmission unit 122 outputs microwaves from the transmission antenna 124 to irradiate the chest of the subject with the microwaves (S101). The microwaves transmitted through the chest of the subject are received by the reception antenna 126. The received microwaves are amplified by the reception unit 128 and are input to the cardiac waveform measurement unit 112 (S102).

The cardiac waveform measurement unit 112 measures the apnea component waveform as illustrated in FIG. 5A from the input microwaves (S103). When being irradiated with the microwaves, the subject stops breathing, and thus the cardiac waveform measurement unit 112 measures only the apnea component waveform 212, for example, in the reception waveform illustrated in FIG. 2A. Next, the frequency calculation unit 115 calculates a heart rate from the apnea component waveform (S104). Calculation of the frequency is performed using any known method. Then, the frequency calculation unit 115 stores the calculated heart rate in the storage unit 117 (S105). Through the process, a more accurate heart rate can be calculated.

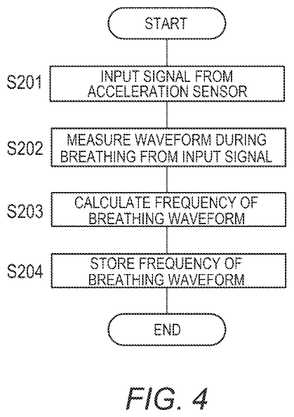

FIG. 4 is an operation flowchart for calculating a breathing frequency by the cardiac output measurement device 100 in accordance with embodiments of the present disclosure. FIG. 5B is a view illustrating a respiratory waveform measured by the respiratory waveform measurement unit 114. In the operation flowchart, a displacement of the chest of the subject during breathing is detected by an acceleration sensor, and from a detected waveform during breathing, a frequency of the waveform during breathing is calculated. Through the process, a more accurate frequency of a waveform during breathing can be calculated.

The respiratory waveform measurement unit 114 inputs a signal from the acceleration sensor 130 (S201). The respiratory waveform measurement unit 114 measures a respiratory waveform of the subject from the input signal (S202). For example, the respiratory waveform measured by the respiratory waveform measurement unit 114 is the waveform as illustrated in FIG. 5B. Next, the frequency calculation unit 115 calculates a frequency of the respiratory waveform (S203). Calculation of the frequency is performed by using a known method that is typically used. Then, the frequency calculation unit 115 stores the calculated frequency of the respiratory waveform in the storage unit 117 (S204).

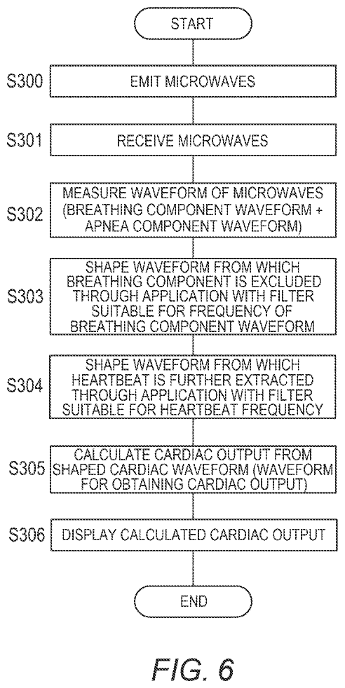

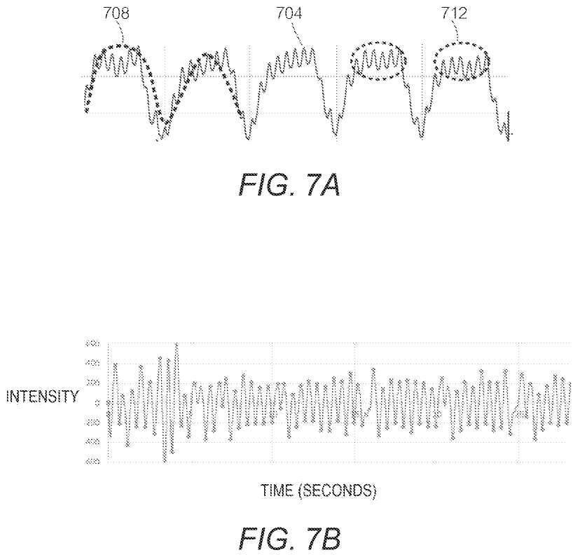

FIG. 6 is an operation flowchart for calculating the cardiac output by the cardiac output measurement device 100 according to embodiments of the present disclosure. In the operation flowchart of FIG. 6, the cardiac output of the subject is calculated by using the heart rate and the breathing frequency stored in the operation flowcharts in FIG. 3 and FIG. 4. The operation flowchart will be described with reference to FIG. 7A and FIG. 7B. FIG. 7A is a view illustrating a reception waveform 704 of microwaves received by the reception unit, including a respiratory component waveform 708 and an apnea component waveform 712. FIG. 7B is a view illustrating an example of a shaped cardiac waveform (e.g., a waveform for obtaining the cardiac output) that is used to obtain the cardiac output by the cardiac output calculation unit.

When the measurement start switch 140 is pressed, the control unit 110 instructs the transmission unit 122 to output microwaves, and the transmission unit 122 outputs the microwaves from the transmission antenna 124 to irradiate the chest of the subject with the microwaves (S300). The microwaves transmitted through the chest of the subject are received by the reception antenna 126. The received microwaves are amplified by the reception unit 128 and are input to the cardiac waveform measurement unit 112 (S301).

The cardiac waveform measurement unit 112 measures a reception waveform in which a respiratory component waveform and an apnea component waveform are mixed as illustrated in FIG. 7B from the input microwaves (S302). The reason for this is because measurement of the cardiac waveform is performed while the subject is breathing.

The cardiac output calculation unit 116 generates a filter suitable for the breathing frequency by using the frequency of the waveform during breathing which is stored in the operation flowchart in FIG. 4, and applies the generated filter to shape a waveform from which the breathing component is excluded (S303). Next, the cardiac output calculation unit 116 generates a filter suitable for the heart rate by using the frequency of the apnea component waveform which is stored in the operation flowchart in FIG. 3, and applies the generated filter to shape a waveform obtained by further extracting a heartbeat (S304).

The cardiac waveform in FIG. 7A is shaped into the cardiac waveform illustrated in FIG. 7B through the process in step 5302 to the process in step 5304. The cardiac output calculation unit 116 calculates the cardiac output of the heart of the subject from the shaped cardiac waveform (waveform for obtaining the cardiac output) illustrated in FIG. 7B (S305). The control unit 110 causes the display unit 154 to display the calculated cardiac output (S306).

As described above, the cardiac output measurement device 100 measures the cardiac output. Note that, in the above-described example, the cardiac output is displayed by the display unit 154, but the cardiac output may be stored in the storage unit 117 or may be transmitted to the external terminal 170 through the communication unit 118.

In addition to the cardiac output, a stroke volume calculated from one waveform amplitude intensity may also be displayed by the display unit 154. Additionally or alternatively, the heart rate may be displayed by the display unit 154. Furthermore, a body surface area may be calculated from information such as an input height and an input weight of the subject, and the cardiac output may be divided by the body surface area to be displayed as a cardiac index.

Note that, in calculation of the stroke volume, the stroke volume may be calculated from the one waveform amplitude intensity. In some embodiments, the cardiac output may be calculated from a cardiac waveform having a plurality of amplitudes, and the value may be divided by a heart rate to calculate the stroke volume.

In some embodiments, the heart rate may be obtained from the apnea component waveform in the operation flowchart in FIG. 3, the breathing frequency may be obtained in the operation flowchart in FIG. 4, and the cardiac output may be obtained by using the frequencies in the operation flowchart in FIG. 6. In some embodiments, the three operation flowcharts may be automatically performed as a series of processes. In addition, in the operation flowchart in FIG. 6, a process of obtaining the frequency of the apnea component waveform and the frequency of the waveform during breathing may be performed to obtain the cardiac output. When obtaining the cardiac output in the operation flowchart in FIG. 6, the frequency of the apnea component waveform and the frequency of the waveform during breathing may be obtained from the cardiac waveform in FIG. 7B.

When employing the configuration in which the three operation flowcharts in FIG. 3, FIG. 4, and FIG. 6 are automatically performed as a series of processes, even in a case where a living body state of the subject varies during a series of measurement operations and a heart rate or a breathing state varies, the cardiac output can be accurately calculated.

Next, a cardiac output measurement method will be described in accordance with at least one embodiment of the present disclosure. The cardiac output measurement method is a method in which the cardiac output is obtained by a series of processes of obtaining the heart rate from the apnea component waveform in the operation flowchart in FIG. 3, obtaining the breathing frequency in the operation flowchart in FIG. 4, and obtaining the cardiac output by using the frequencies in the operation flowchart in FIG. 6.

First, a cardiac waveform composed of an apnea component waveform is measured from microwaves transmitted through the chest of the subject (as shown in the operation flowchart in FIG. 3).

Next, a heart rate is calculated from the apnea component waveform (as shown in the operation flowchart in FIG. 3).

Next, a waveform during breathing or a waveform during apnea is measured from a displacement of a body surface (e.g., the chest) of the subject (as shown in the operation flowchart in FIG. 4).

Next, a breathing frequency is calculated (as shown in the operation flowchart in FIG. 4).

Next, a cardiac waveform including both the respiratory component waveform and the apnea component waveform is measured from microwaves transmitted through the chest of the subject (as shown in the operation flowchart in FIG. 6).

Next, a filter is created by using the heart rate and the breathing frequency, and the filter is applied for shaping the cardiac waveform (as shown in the operation flowchart in FIG. 6).

Finally, the cardiac output is calculated from the shaped cardiac waveform (as shown in the operation flowchart in FIG. 6).

As described above, according to embodiments of the present disclosure, an influence by breathing of the subject is removed, and thus accurate measurement of the cardiac output can be performed even in a state in which the subject is breathing.

In the cardiac output measurement device and the cardiac output measurement method, the frequency of the waveform during breathing which is obtained from the acceleration sensor 130, and the frequency of the apnea component waveform obtained from the microwaves are individually calculated. However, for example, the cardiac waveform (the respiratory component waveform and the apnea component waveform) obtained from the microwaves may be Fourier-transformed to separate the respiratory component waveform and the apnea component waveform from each other, thereby calculating the frequency of each of the respiratory component waveform and the apnea component waveform.

While measurement of the cardiac output has been described herein, an index such as the stroke volume and the cardiac index is present and may be determined using embodiments discussed herein. Furthermore, and the indexes are related and can be converted to each other (e.g., from stroke volume to cardiac index and vice versa), and thus the indexes are not particularly limited.

In some embodiments, electromagnetic waves within the microwave frequency range (e.g., between about 300 Megahertz (MHz) and about 300 Gigahertz (GHz)), such as microwaves having a frequency of 0.4 to 1.00 GHz, may be used. However, with regard to a definition of the microwaves, there is no difference between a definition of electromagnetic waves having a frequency of 300 MHz to 300 GHz and a definition of electromagnetic waves having a frequency of 3 GHz to 30 GHz. In addition, it is preferable to set a frequency at which a waveform for obtaining the cardiac output is most clearly obtained, and electromagnetic waves with alternative frequencies and wavelengths may be used.

According to embodiments of the present disclosure, in measurement of the cardiac output, satisfactory measurement accuracy can be expected. Additionally, it is also possible to continuously monitor the cardiac output of a patient who is in poor condition in an intensive care unit or the like.

It is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

What is claimed is:

1. A cardiac output measurement device, comprising:
a first measurement unit that measures a first waveform of microwaves transmitted through a living body;
a second measurement unit that measures a second waveform during breathing or a third waveform during apnea of the living body; and
a calculation unit that calculates a fourth waveform for obtaining a cardiac output of the living body from the first waveform of the microwaves by using the second waveform during breathing or the third waveform during apnea.

2. The device of claim 1, wherein the first measurement unit further includes a cardiac waveform measurement unit that measures a fifth waveform including an apnea component waveform from the first waveform of the microwaves transmitted through the living body.

3. The device of claim 2, wherein the second measurement unit includes a respiratory waveform measurement unit that measures the second waveform during breathing from a displacement of a body surface of the living body.

4. The device of claim 3, wherein an acceleration sensor attached to the body surface of the living body is connected to the respiratory waveform measurement unit.

5. The device of claim 2, wherein the calculation unit includes:
a frequency calculation unit that calculates a first frequency of the second waveform during breathing and a second frequency of the fifth waveform including the apnea component waveform; and
a cardiac output calculation unit that calculates the cardiac output by shaping a cardiac waveform from the fifth waveform measured by the cardiac waveform measurement unit by using at least one of the first frequency or the second frequency.

6. The device of claim 5, wherein the cardiac output calculation unit generates a first filter capable of removing the first frequency and a second filter capable of removing the second frequency included in the cardiac waveform, and wherein the cardiac output calculation unit shapes the cardiac waveform by applying the first filter and the second filter to the cardiac waveform.

7. The device of claim 6, further comprising:
a storage unit that stores a plurality of filter coefficient calculation formulas and a plurality of filters,
wherein the cardiac output calculation unit generates the first filter and the second filter from the plurality of filter coefficient calculation formulas and the plurality of filters.

8. The device of claim 7, wherein the plurality of filters includes digital filters, and wherein the digital filters include at least one of a low-pass filter or a bandpass filter.

9. The cardiac output measurement device of claim 2, further comprising:
a notification unit that generates a message for promoting stoppage of breathing of the living body.

10. A cardiac output measurement method, comprising:
measuring a cardiac waveform including an apnea component waveform created by transmitting microwaves through a living body;
calculating a first frequency of the apnea component waveform;
measuring a first waveform during breathing from a displacement of a body surface of the living body;
calculating a second frequency of the first waveform during breathing;
measuring a second waveform including both a respiratory component waveform and the apnea component waveform from the microwaves transmitted through the living body;
shaping the cardiac waveform by using the first frequency of the apnea component waveform and the second frequency of the first waveform during breathing; and
calculating a cardiac output from the shaped cardiac waveform.

11. The method of claim 10, further comprising:
generating a first filter capable of removing the first frequency of the apnea component waveform; and
generating a second filter capable of removing the second frequency of the first waveform during breathing.

12. The method of claim 11, further comprising:
generating the first filter and the second filter from a plurality of filter coefficient calculation formulas and a plurality of filters.

13. The method of claim 12, further comprising:
applying the first filter and the second filter to the cardiac waveform.

14. The method of claim 13, wherein the plurality of filters includes a plurality of digital filters.

15. The method of claim 14, wherein the plurality of digital filters includes at least one of a low-pass filter or a bandpass filter.

16. The method of claim 10, further comprising:
generating a notification instructing a patient to stop breathing.

17. A cardiac output measurement device, comprising:
a cardiac waveform measurement unit that measures a microwave waveform transmitted through a patient, the microwave waveform including both a respiratory waveform component and an apnea waveform component;
a respiratory waveform measurement unit that measures a second waveform generated while the patient is breathing;
a calculation unit that calculates a cardiac output from the microwave waveform, the calculation unit including:
a frequency calculation unit that determines at least one frequency of the second waveform, and
a cardiac output calculation unit that generates a first filter based on the at least one frequency, shapes a cardiac waveform by applying the first filter to the microwave waveform, and calculates the cardiac output based on the shaped cardiac waveform.

18. The device of claim 17, wherein the frequency calculation unit determines a second frequency of the apnea waveform component, and wherein the cardiac output calculation unit generates a second filter based on the second frequency.

19. The device of claim 18, wherein shaping the cardiac waveform further includes the cardiac output calculation unit applying the second filter to the microwave waveform.

20. The device of claim 17, further comprising:
an acceleration sensor disposed on the patient and connected to the respiratory waveform measurement unit.
